(19) United States
(12) Patent Application Publication
(10) Pub. No.: US 2011/0224413 A1
(43) Pub. Date: Sep. 15, 2011

(54) CHEMICAL PROCESS

(76) Inventors: Michael Tolar Martin, Durham, NC (US); Michael S. McClure, Durham, NC (US); Vassil Elitzin, Durham, NC (US)

(21) Appl. No.: 13/129,496

(22) PCT Filed: Nov. 19, 2009
(86) PCT No.: PCT/US09/65061

§ 371 Date: May 16, 2011

Related U.S. Application Data
(60) Provisional application No. 61/116,658, filed on Nov. 21, 2008.

(51) Int. Cl. C07H 17/02; C07D 231/20

(52) U.S. Cl. 536/17.4; 548/366.1

(57) ABSTRACT

Disclosed herein are processes for preparing glucopyranosyloxypyrazole derivatives and pyrazole intermediates of the same. In particular, the present invention relates to glucopyranosyloxypyrazole derivatives having SGLT2 inhibitory activity and processes and intermediates for preparing the same.

### BACKGROUND OF THE INVENTION

The present invention relates to processes for preparing glucopyranosyloxypyrazole derivatives and pyrazole intermediates useful in said processes. In particular, the present invention relates to glucopyranosyloxypyrazole derivatives having SGLT2 inhibitory activity and processes and intermediates for preparing the same.

Sodium dependent glucose transporters (SGLT), including SGLT1 and SGLT2, are membrane proteins that transport glucose. SGLT2 is mainly active in the proximal tubules of the kidney wherein it affects the transport of glucose from the urine into the bloodstream. The reabsorbed glucose is then utilized throughout the body. Diabetic patients are typically characterized by abnormal blood glucose levels. Consequently, inhibition of SGLT2 activity and therefore inhibition of glucose reabsorption in the kidneys is believed to be a possible mechanism for controlling blood glucose levels in such diabetic patients. Glucopyranosyloxypyrazole derivatives have been proposed for treatment of diabetic patients, with some being currently in clinical development. See U.S. Pat. Nos. 6,972,283; 7,056,892; 7,084,123; 7,393,838; 7,429,568; 6,815,428; 7,015,201; 7,247,616; and 7,256,209. Accordingly, scalable and cost efficient synthesis of glucopyranosyloxypyrazole derivatives as well as intermediates for producing the same is a current need in the pharmaceutical industry.

### BRIEF SUMMARY OF THE INVENTION

The present inventors have now discovered processes for preparing glucopyranosyloxypyrazole derivatives, intermediates for use in the same, as well as processes for producing said intermediates.

In one aspect of the present invention, there is provided a process for preparing a compound of formula (II),

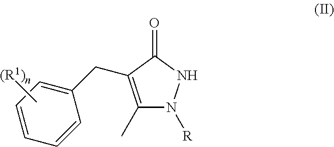

comprising the steps of:
(i) O-sulfonating a compound of formula (Ia)

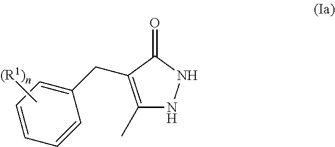

to produce a compound of formula (Ib);

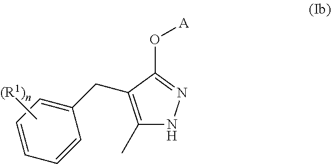

(ii) alkylating the compound of formula (Ib) to produce a compound of formula (Ic); and

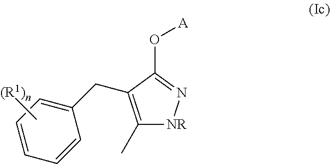

(iii) desulfonating the alkylated compound of formula (Ic) to produce the compound of formula (II);
   wherein:
   R is C₁-C₆ alkyl;
   n is 0-3,
   R¹ is C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo; and
   A is a sulfonyl or sulfinyl containing hydroxyl protecting group.

In a second aspect of the present invention, there is provided a process for preparing a compound of formula (III),

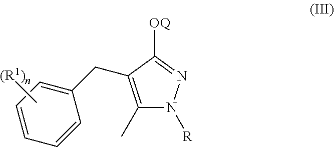

comprising the steps of:
(i) O-sulfonating a compound of formula (Ia)

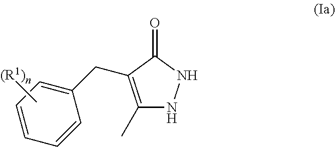

to produce a compound of formula (Ib);

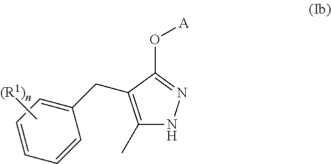

(iii) alkylating the compound of formula (Ib) to produce a compound of formula (Ic);

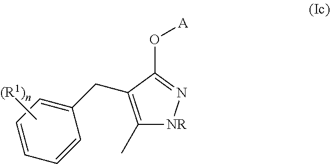

(iii) desulfonating the alkylated compound of formula (Ic) to produce the compound of formula (II); and

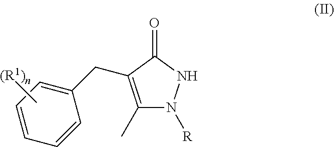

(iv) reacting a compound of formula (II) with a glucose derivative to provide a compound of formula (III),
   wherein:
   R is C₁-C₆ alkyl;
   n is 0-3,
   R¹ is C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo;
   A is a sulfonyl or sulfinyl containing hydroxyl protecting group; and
   wherein Q is:

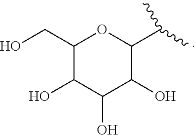

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” refers to a straight or branched chain hydrocarbon, e.g., from one to twelve carbon atoms. Examples of “alkyl”, as used herein include, but are not limited to, methyl, ethyl, n-propyl, isopropyl, n-butyl, n-pentyl, and isobutyl, and the like.

As used herein, the term “C₁-C₆ alkyl” refers to an alkyl group, as defined above, which contains at least 1, and at most 6, carbon atoms. Examples of “C₁-C₆ alkyl” groups useful in the present invention include, but are not limited to, methyl, ethyl, n-propyl, isopropyl, isobutyl and n-butyl.

As used herein, the term “alkenyl” refers to a hydrocarbon group, e.g., from two to ten carbons, and having at least one carbon-carbon double bond. Examples of “alkenyl”, as used herein include, vinyl (ethenyl), propenyl, 2-methyl-1-propenyl, 1-butenyl, 2-butenyl, and isobutenyl.

As used herein, the term “C₂-C₆ alkenyl” refers to an alkenyl group, as defined above, containing at least 2, and at most 6, carbon atoms. Examples of “C₂-C₆ alkenyl” groups useful in the present invention include, but are not limited to, vinyl (ethenyl), propenyl, 2-methyl-1-propenyl, 1-butenyl, 2-butenyl, and isobutenyl.

As used herein, the term “alkynyl” refers to a hydrocarbon group, e.g., from two to ten carbons, and having at least one carbon-carbon triple bond. Examples of “alkynyl”, as used herein, include but are not limited to ethynyl (acetylenyl), 1-propynyl, 1-butynyl, 2-butynyl, 1-pentynyl, and 1-hexynyl.

As used herein, the term “C₂-C₆ alkynyl” refers to an alkynyl group, as defined above, containing at least 2, and at most 6, carbon atoms. Examples of “C₂-C₆ alkynyl” groups useful in the present invention include, but are not limited to, ethynyl (acetylenyl), 1-propynyl, 1-butynyl, 2-butynyl, 1-pentynyl, and 1-hexynyl.

As used herein, the term “acyl” refers to the group RₐC(O)—, where Rₐ is alkyl as defined herein and the term “C₁-C₆ acyl” refers to the group RₐC(O)—, where Rₐ is C₁-C₆ alkyl as defined herein. Examples of “C₁-C₆ acyl” groups useful in the present invention include, but are not limited to, acetyl and propionyl.

As used herein, the terms “halo” refer to fluoro (—F), chloro (—Cl), bromo (—Br), or iodo (—I).

As used herein, the term “C₁-C₆ haloalkyl” refers to an alkyl group, as defined above, containing at least 1, and at most 6, carbon atoms substituted with at least one halo group, halo being as defined herein. Examples of “C₁-C₆ haloalkyl” groups useful in the present invention include, but are not limited to, methyl, ethyl, n-propyl, isopropyl, isobutyl and n-butyl substituted independently with one or more halo groups, e.g., fluoro, chloro, bromo and iodo.

As used herein, the term “alkoxy” refers to the group RₐO—, where Rₐ is alkyl as defined above and the term “C₁-C₆ alkoxy” refers to the group RₐO—, where Rₐ is C₁-C₆ alkyl as defined above. Examples of “C₁-C₆ alkoxy” groups useful in the present invention include, but are not limited to, methoxy, ethoxy, propyloxy, and isopropyloxy.

As used herein the term “C₁-C₆ haloalkoxy” refers to the group RₐO—, where Rₐ is C₁-C₆ haloalkyl as defined above. An exemplary C₁-C₆ haloalkoxy group useful in the present invention includes, but is not limited to, trifluoromethoxy.

As used herein, the term “alkylthio” refers to the group RₐS—, where Rₐ is alkyl as defined above and the term “C₁-C₆ alkylthio” refers to the group RₐS—, where Rₐ is C₁-C₆ alkyl as defined above. Examples of “C₁-C₆ alkylthio” groups useful in the present invention include, but are not limited to, methylthio, ethylthio, and propylthio.

As used herein, the term “C₁-C₆ haloalkylthio” refers to the group RₐS—, where Rₐ is C₁-C₆ haloalkyl as defined above. Examples of “C₁-C₆ haloalkylthio” groups useful in the present invention include, but are not limited to, methylthio, ethylthio, and propylthio wherein the alkyl is substituted independently with one or more halo groups, e.g., fluoro, chloro, bromo and iodo.

As used herein the term “C₁-C₆ alkylamino” refers to the group —NRₐR_{b} wherein Rₐ is —H or C₁-C₆ alkyl and R_{b} is —H or C₁-C₆ alkyl, where at least one of Rₐ and R_{b} is C₁-C₆alkyl and C₁-C₆ alkyl is as defined above. Examples of “C₁-C₆ alkylamino” groups useful in the present invention include, but are not limited to, methylamino, ethylamino, propylamino, dimethylamino, and diethylamino.

As used herein, the term “C₃-C₇ cycloalkyl” refers to a non-aromatic hydrocarbon ring having from three to seven carbon atoms, which may or may not include a C₁-C₄ alkylene linker, through which it is attached, said linker being attached directly to the ring. Exemplary “C₃-C₇ cycloalkyl” groups include, but are not limited to, cyclopropyl, cyclobutyl, cyclopentyl, cyclohexyl, cycloheptyl, and cyclopropylmethylene.

As used herein, the term “C₃-C₇ cycloalkyloxy” refers to the group RₐO—, where Rₐ is C₃-C₇ cycloalkyl as defined above. Examples of “C₃-C₇ cycloalkyloxy” groups useful in the present invention include, but are not limited to, cyclopropyloxy, cyclobutyloxy, and cyclopentyloxy.

As used herein, the term “aryl” refers to a benzene ring or to a benzene ring system fused to one or more benzene or heterocyclyl rings to form, for example, anthracene, phenanthrene, napthalene, or benzodioxin ring systems. Examples of “aryl” groups include, but are not limited to, phenyl, 2-naphthyl, 1-naphthyl, biphenyl, 1,4-benzodioxin-6-yl as well as substituted derivatives thereof.

The present invention includes a process for preparing a compound of formula

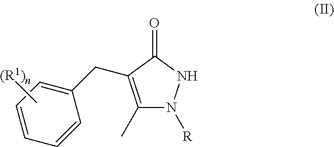

In one embodiment, R is C₁-C₆ alkyl. In another embodiment, R is methyl, ethyl, n-propyl, isopropyl, and n-butyl. In one embodiment, R is isopropyl.

In one embodiment, n is 0-3. In another embodiment, n is 1 or 2. In one embodiment, n is 1. In another embodiment, n is 2.

In one embodiment, R¹ is C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo.

In another embodiment, R¹ is C₁-C₆ alkyl, C₁-C₆ alkoxy, C₁-C₆ alkylthio, C₁-C₆ haloalkyl, or halo. In another embodiment, R¹ is C₁-C₆ alkyl, C₁-C₆ alkoxy, or halo.

In one embodiment, n is 1 and R¹ is isopropoxy. In another embodiment, n is 2 and at least one of R¹ is halo. In another embodiment, n is 2 and at least one of R¹ is fluoro.

In one embodiment n is 1 and R¹ is attached at the para position of the phenyl. In one embodiment n is 1 and R¹ is attached at the ortho position of the phenyl. In one embodiment n is 1 and R¹ is attached at the meta position of the phenyl.

In one embodiment, R is methyl, ethyl, n-propyl, isopropyl, and n-butyl; n is 1 or 2; and each R¹ is independently selected from C₁-C₆ alkyl, C₁-C₆ alkoxy, or halo.

In another embodiment, R is methyl, ethyl, n-propyl, isopropyl, and n-butyl; n is 1; and R¹ is C₁-C₆ alkyl, C₁-C₆ alkoxy, or halo.

In another embodiment, R is methyl, ethyl, n-propyl, isopropyl, and n-butyl; n is 2; and each R¹ is independently selected from C₁-C₆ alkyl, —C₁-C₆ alkoxy, or halo.

In one embodiment, R is isopropyl and R¹ is isopropoxy.

In one embodiment, R is isopropyl and R¹ is isopropoxy, wherein the isopropoxy group is attached at the para position of the phenyl group.

In another embodiment, R is isopropyl, n is 2 and at least one R¹ is halo.

In another embodiment, R is isopropyl, n is 2 and at least one R² is fluoro.

In another embodiment, R is isopropyl, n is 2 and one R² is halo and the other is C₁-C₆ alkoxy.

In another embodiment, R is isopropyl, n is 2 and one R² is fluoro and the other is methoxy.

In another embodiment, R is isopropyl, n is 2 and one R² is halo and the other is C₁-C₆ alkyl.

In another embodiment, R is isopropyl, n is 2 and one R² is fluoro and the other is methyl.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted.

The presence of a double bond is possible in the compounds described herein, accordingly also included in the compounds of the invention are their respective pure E and Z geometric isomers as well as mixtures of E and Z isomers. The invention as described and claimed does not set any limiting ratios on prevalence of Z to E isomers.

The compound of formula (II) is prepared by O-sulfonating a compound of formula (Ia)

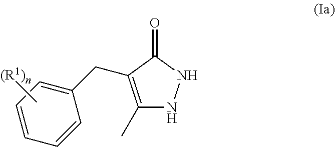

to provide a compound of formula (Ib);

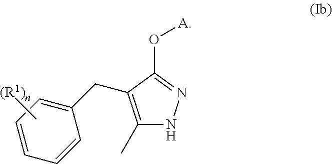

R¹ and n are as defined above.

As recited above A is a sulfonyl or sulfinyl containing hydroxyl protecting group.

In one embodiment, A is a group

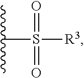

which is derived from the sulfonyl halide following:

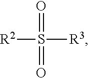

wherein

### R²=—Cl, —Br, or —F;

R³═C₁-C₆ alkyl, C₃-C₇ cycloalkyl, or phenyl substituted with R⁴;
where R⁴=—H, —Cl, —Br, —F, —NO₂, alkyl, cycloalkyl, or —OR⁵; and
where R⁵═C₁-C₆ alkyl or C₃-C₇ cycloalkyl.

In another embodiment, A is a group

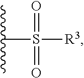

which is derived from the sulfonyl anhydride following:

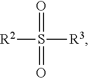

wherein
R²═RS(O)₂O—, where R═C₁-C₆ alkyl, C₃-C₇ cycloalkyl, or phenyl substituted with R⁴;
R³═C₁-C₆ alkyl, C₃-C₇ cycloalkyl, or phenyl substituted with R⁴;
where R⁴=—H, —Cl, —Br, —F, —NO₂, alkyl, cycloalkyl, or —OR⁵; and
where R⁵═C₁-C₆ alkyl or C₃-C₇ cycloalkyl.

In another embodiment, A is a group

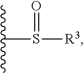

which is derived from the sulfinyl halide following:

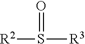

wherein R² is —Cl, —Br, or —F and R³ is as defined above.

The O-sulfonation of the compound of formula (Ia) is typically carried out utilizing a sulfonyl halide in the presence of a base in a suitable solvent. Scheme 1 depicts two embodiments of such a sulfonation—tosylation and mesylation.

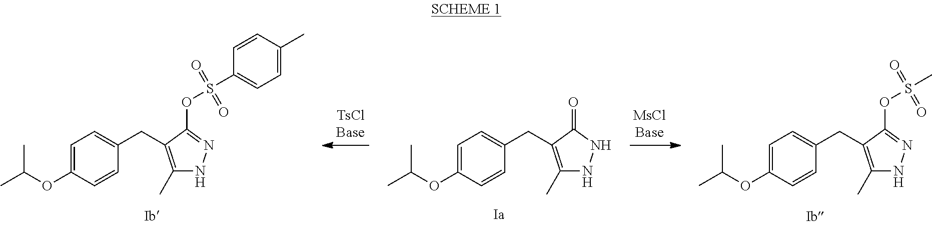

Scheme 1 illustrates the tosylation and mesylation of a compound of formula (Ia), wherein R¹ is isopropoxy and n is 1, to give sulfonated compounds of formula Ib′ and Ib″. These sulfonated compounds are the tosylated and mesylated forms of the specific compounds of formula (Ia) respectively. Tosylation of the compound of formula (Ia) was performed by reaction with tosyl chloride optionally in the presence of a base in a suitable solvent. The typical temperature range utilized was 15-30° C. Suitable solvents include, but are not limited to, N,N-dimethylformamide (DMF), acetonitrile (MeCN), dichloromethane (CH₂Cl₂), and ethyl acetate (EtOAc). Bases which may be utilized include, but are not limited to, cesium carbonate (Cs₂CO₃), potassium carbonate (K₂CO₃), pyridine, and triethylamine (Et₃N). Mesylation of the compound of formula (Ia) was performed by reaction with methanesulfonyl chloride or methanesulfonic anhydride optionally in the presence of a base in a suitable solvent. Suitable solvents include, but are not limited to, N,N-dimethylformamide, (DMF), acetonitrile (MeCN), and n-methyl pyrrolidinone (NMP). Bases which may be utilized include, but are not limited to, pyridine, triethylamine (Et₃N), and lithium hydroxide (LiOH). Isolatable solids are obtainable for both tosyl and mesyl intermediates. Mono-sulfonation is obtained by using no added base or a very weak base such as pyridine. Accordingly, in one embodiment, the tosylation or mesylation takes place in the presence of a weak base, for instance pyridine. In another embodiment, the tosylation or mesylation takes place without use of a base. The O-sulfonated intermediates of formula (Ib′) and (Ib″) alkylate on nitrogen with good regioselectivity. Typically regioselectivity of about 10:1 is observed.

The O-sulfonated compound of formula (Ib), for example the compound of formula (Ib′) or (Ib″), is then alkylated to form a compound of formula I(c) and then the compound of formula I(c) is deprotected (desulfonated) to form a compound of formula (II). In this instance R¹ is isopropoxy, n is 1, and R is isopropyl. Scheme 2 depicts the alkylation (isopropylation) and deprotection of the compound of formula (Ib′), i.e., the tosyl protected intermediate.

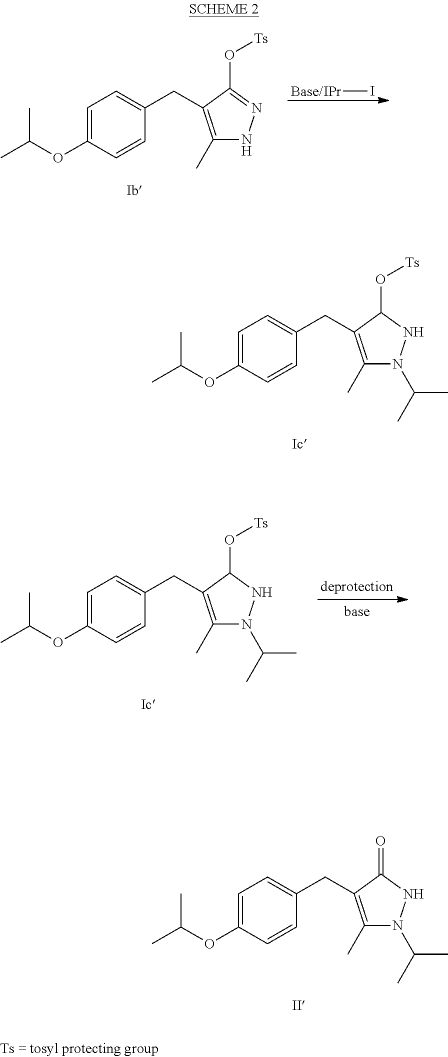

Alkylation of the compound of formula (Ib′) proceeds with reaction with an alkyl halide, for instance isopropyl iodide, in the presence of a base in a suitable solvent. The alkylation reaction is typically run at 20-30° C. Bases which may be utilized include, but are not limited to, potassium carbonate (K₂CO₃), 1,8-diazabicyclo[5.4.0]undec-7-ene (DBU), potassium tert-butoxide (KOtBu), triethylamine (Et₃N), lithium hydroxide (LiOH), cesium carbonate (Cs₂CO₃), sodium tert-butoxide (NaOtBu), potassium hydroxide (KOH), and pyridine). Suitable solvents include N,N-dimethylformamide (DMF), acetonitrile (MeCN), dichloromethane (CH₂Cl₂). Ratios achieved are on the order of 10:1 regioselectivity. Decomposition of excess alkyl halide via reaction with ethanolamine or other nucleophile may be performed prior to deprotection of O-sulfonate. Deprotection (desulfonation) proceeds by reaction with a base, such as NaOH, at a temperature of about 60-70° C. to arrive at the compound of formula II′.

Scheme 3 depicts alkylation and deprotection of the compound of formula (Ib″), i.e., the mesyl protected intermediate.

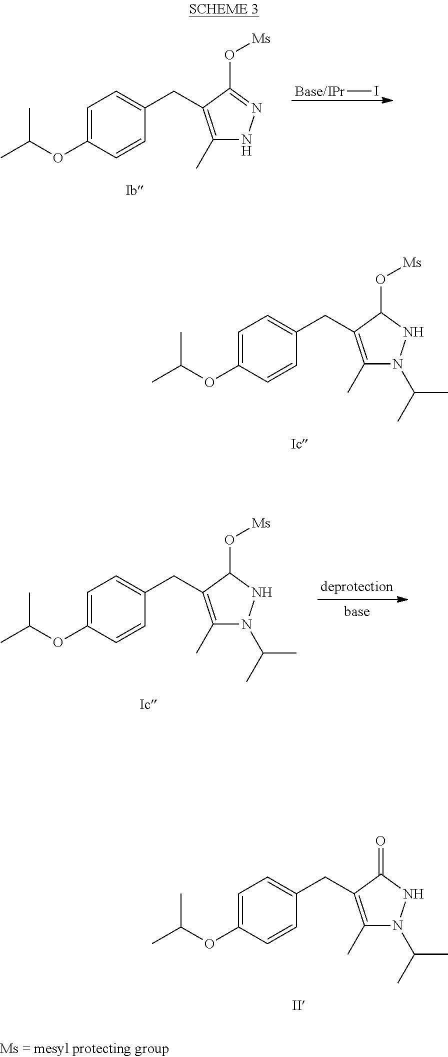

Alkylation of the compound of formula (Ib″) proceeds with reaction with an alkyl halide, for instance isopropyl iodide, in the presence of a base in a suitable solvent. The alkylation reaction is typically run at 20-30° C. Usable bases include, but are not limited to, lithium hydroxide (LiOH), sodium hydroxide (NaOH), potassium hydroxide (KOH), potassium tert-butoxide (KOtBu), cesium carbonate (Cs₂CO₃), potassium carbonate (K₂CO₃), sodium tert-butoxide (NaOtBu), lithium tert-butoxide (LiOtBu), lithium carbonate (Li₂CO₃), and sodium carbonate (Na₂CO₃). Suitable solvents include, but are not limited to, N,N-dimethylformamide (DMF), N-methylpyrrolidinone (NMP), N,N-dimethylacetamide (DMAC) and acetonitrile (MeCN). Prior to deprotection, decomposition of excess alkyl halide via reaction with ethanolamine or other nucleophile may be performed prior to deprotection of O-sulfonate. Deprotection (desulfonation) proceeds by reaction with a base, such as NaOH, at a temperature of about 60-70° C. to arrive at the compound of formula II″.

Typical alkylating agents which may be utilized to effect the alkylation of the starting compounds of Schemes 2 or 3 are alkyl halides. Specific alkylating agents for isopropylation of the starting compounds of Schemes 2 and 3, including isopropyl halides, may be as follows:

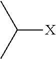

where X is —Cl, —F, —Br, —I, or —OR⁶ where R⁶ is mesyl, tosyl, or nosyl.

In one embodiment, the alkylating agent is isopropyl iodide.

In one embodiment, the alkylation reaction is quenched with a mild base, for example, ethanolamine to destroy the remaining isopropyl iodide prior to deprotection in order to protect against bis-alkylation.

Typical mild bases which may be utilized to quench the alkylation reaction to avoid bis-alkylation, include compounds of the following structures:

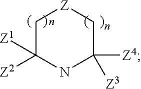

wherein:
Z¹, Z², Z³, and Z⁴ are independently H, C₁-C₆ alkyl, O₃—C₇ cycloalkyl, or aryl,
Z is CH₂, N, O, or S, and
n is 0 to 3;

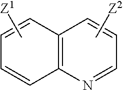

wherein:
Z¹ and Z² are independently selected from —H, C₁-C₆ alkyl, aryl, C₃-C₇ cycloalkyl, —F, —Cl, and —Br;

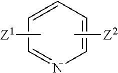

Z¹ and Z² are independently selected from —H, C₁-C₆ alkyl, aryl, C₃-C₇ cycloalkyl, —F, —Cl, and —Br;

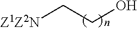

Z¹ and Z² are independently selected from —H, C₁-C₆ alkyl, C₃-C₇ cycloalkyl, and aryl,
n is 0 to 3;

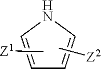

Z¹ and Z² are independently selected from —H, C₁-C₆ alkyl, aryl, C₃-C₇ cycloalkyl, —F, —Cl, or —Br;

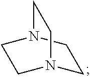

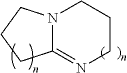

n is 0 to 3;
   and
Z¹Z²Z³N wherein Z¹, Z², Z³ are independently selected from —H, C₁-C₆ alkyl, C₃-C₇ cycloalkyl, or aryl.

Once prepared, the compound of formula (II) may be glyclosidated to form a compound of formula (III):

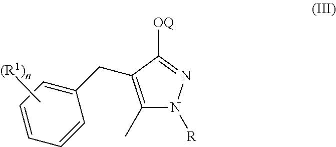

wherein Q is:

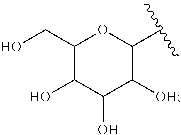

and R, R¹ and n are as defined above.

In one embodiment Q is:

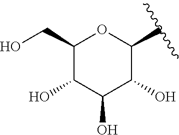

Scheme 4 depicts one embodiment of such a glucosidation.

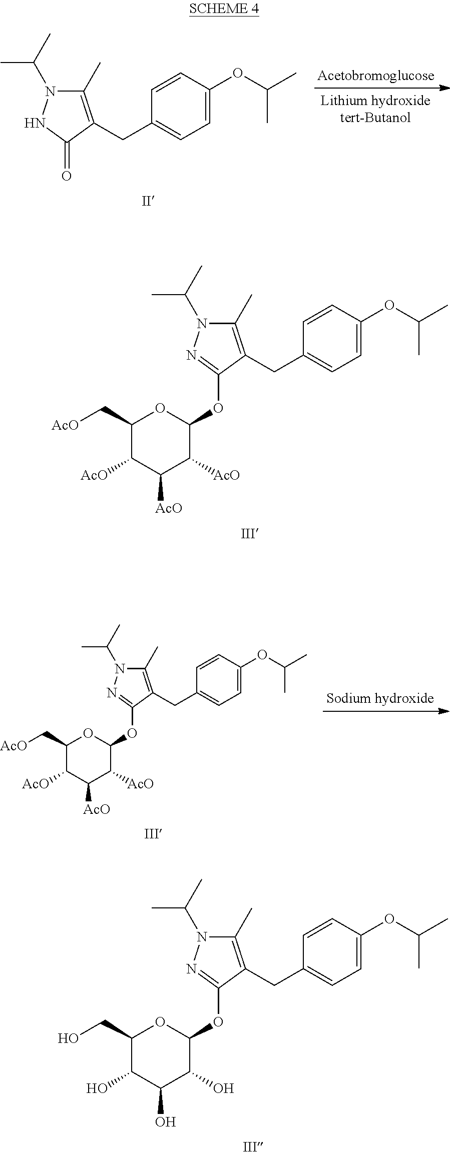

The glucosidation or glycosylation of the compound of formula II, in this embodiment a compound of Formula II′, is typically carried out using a protected and anomerically activated glucose derivative in the presence of a base in a suitable solvent to form a compound of Formula III′. The compound of formula III′ is then hydrolyzed with a strong base, such as sodium hydroxide, to cleave the acetyl protecting groups to arrive at the compound of formula III″ Both reactions are carried out at a temperature of about 35 to 40° C. Protecting groups which may be utilized include, but are not limited to, acetyl and pivaloyl. Activating groups which may be utilized include, but are not limited to chloride and bromide. Inorganic bases which may be utilized include, but are not limited to, sodium hydride, lithium hydroxide, sodium hydroxide, potassium hydroxide, cesium hydroxide, lithium carbonate, sodium carbonate, potassium carbonate, and cesium carbonate. Organic bases which may be utilized include, but are not limited to lithium tert-butoxide, sodium tert-butoxide, potassium tert-butoxide, tert-butyl lithium, lithium diisopropyl amide, and lithium hexamethyldisilazane. Suitable solvents which may be utilized include, but are not limited to toluene, acetone, 2-butanone, methyl-isobutyl ketone, ethanol, methanol, isopropanol, butanol, tert-butanol, neopentanol, tetrahydrofuran, 2-methyl tetrahydrofuran, methyl tert-butyl ether, and dichloromethane. The glycosidation is very selective for the O-position of compound II.

In another embodiment, there is provided a compound useful as an intermediate in the preparation of compounds of formula (II):

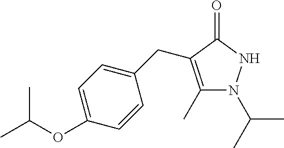

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| N (normal); | Hz (Hertz); |
| Vol (volumes) | MHz (megahertz); |
| mol (moles); | mmol (millimoles); |
| RT (room temperature); | RP (reverse phase); |
| min (minutes); | h (hours); |
| mp (melting point); | TLC (thin layer chromatography); |
| Tᵣ (retention time); | MeOH (methanol); |
| I-PrOH (isopropanol); | HOAc (acetic acid); |
| TEA (triethylamine); | TFA (trifluoroacetic acid); |
| THF (tetrahydrofuran); | NMP (n-methylpyrrolidinone) |
| DMSO (dimethylsulfoxide); | EtOAc (ethyl acetate); |
| DME (1,2-dimethoxyethane); | DCM (dichloromethane); |
| DCE (dichloroethane); | DMF (N,N-dimethylformamide); |
| atm (atmosphere); | |
| HPLC (high pressure liquid | |
| chromatography); | |

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, a Varian VNMRS-500, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), h (heptet), q (quartet), m (multiplet), br (broad).

Low-resolution mass spectra (MS) were recorded on a JOEL JMS-AX505HA, JOEL SX-102, Agilent series 1100MSD, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60E-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

The following examples describe the syntheses of intermediates particularly useful in the synthesis of compounds of Formula (I):

### Example 1

### 5-methyl-1-(1-methylethyl)-4-({4-[(1-methylethyl)oxy]phenyl}methyl)-1,2-dihydro-3H-pyrazol-3-one (3) Brackets Formula III

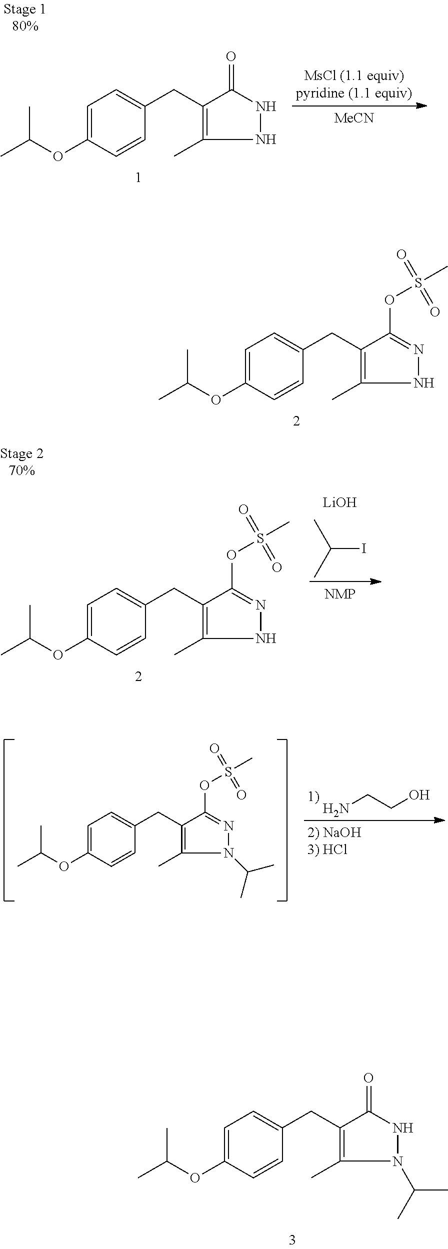

### (i) Preparation of 5-methyl-4-({4-[(1-methylethyl)oxy]phenyl}methyl)-1H-pyrazol-3-yl methanesulfonate (2)

To a stirred solution of 200 g (0.81 moles) of 5-methyl-4-({4-[(1-methylethyl) oxy]phenyl}methyl)-1,2-dihydro-3H-pyrazol-3-one (1) in acetonitrile (5 vol) at 20° C. was added 102 g (0.89 moles) of methanesulfonyl chloride and 59 g (0.89 moles) of pyridine. The reaction was stirred at 20-25° C. for 1 to 2 hours. Water (15 vol) was added over a period of 20 minutes and the reaction stirred at 15 to 20° C. for 1 hour. Solids are filtered and washed with additional water (2×2-vol) to give 210 g (80%) of the desired compound as an off white solid. ¹H NMR (300 MHz, DMSO) δ 7.04 (d, J=8.8 Hz, 2H), 6.79 (d, J=8.8 Hz, 2H), 4.52 (h, J=6.1 Hz, 1H), 3.58 (s, 2H) 3.44 (s, 3H), 2.08 (s, 3 H), 1.22 (d, J=6.1 Hz, 6H)

### (ii) Preparation of 5-methyl-1-(1-methylethyl)-4-({4-[(1-methylethyl)oxy]phenyl}methyl)-1,2-dihydro-3H-pyrazol-3-one (3)

To a stirred solution of 175 g (0.54 moles) of 5-methyl-4-({4-[(1-methylethyl) oxy]phenyl}methyl)-1H-pyrazol-3-yl methanesulfonate (2) in NMP (5 vol) at 20° C. was added 38.7 g (1.62 moles) of lithium hydroxide and 275 g (1.6 moles) of isopropyl iodide. The contents were stirred at 20 to 25° C. for 2 hours and then 98.9 g (1.6 moles) of ethanolamine was added and the contents stirred at 60° C. for 1 hour. Then, 404 ml (1.6 moles) of 4N NaOH and methanol (5 vol) were added and the reaction mixture was maintained at 60° C. for one hour. The contents were cooled to 15° C. and the pH adjusted to between 7 to 9 by addition of 12 N hydrochloric acid and 200 ml water. The contents were then heated to 60 degrees for ˜5 minutes and then cooled to 15° C. degrees and held for 16 hours. Solids were filtered and washed with water (2×2 vol) and then dried at 60° C. to give the desired title compound as off white solid (108.8 g, 70% yield). ¹H NMR (300 MHz, DMSO) δ 9.41 (s, 1H), 7.03 (d, J=8.6 Hz, 2H), 6.77 (d, J=8.6 Hz, 2H), 4.51 (h, J=6.1 Hz, 1H), 4.28 (h, J=6.6 Hz, 1H), 3.44 (s, 2H), 2.06 (s, 3 H), 1.25 (d, J=6.6 Hz, 6H), 1.21 (d, J=6.1 Hz, 6H).

### Example 2

### Preparation of 5-methyl-1-(1-methylethyl)-4-({4-[(1-methylethyl)oxy]phenyl}methyl)-1H-pyrazol-3-yl β-D-glucopyranoside (4)

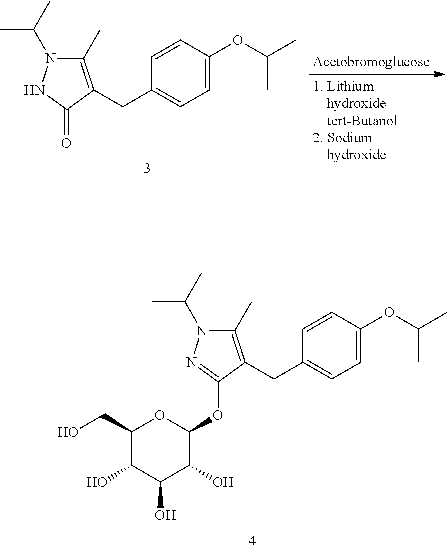

To a stirred mixture of 1500 g (5.20 mol) of 5-methyl-1-(1-methylethyl)-4-({4-[(1-methylethyl)oxy]phenyl}methyl)-1,2-dihydro-3H-pyrazol-3-one (3) in 15 L (10 vol) of tert-Butyl alcohol was added 3200 g (7.80 mol) of 2,3,4,6-tetra-o-acetyl-α-D-glucopyranosyl bromide and 311 g (13 mol) of anhydrous lithium hydroxide powder. The reaction was heated to 38° C. for 4 hours. To this mixture was charged 721 g (33.8 mol) of 25% w/w sodium hydroxide solution and the reaction temperature adjusted to 38° C. and held for 1 hour. Charged 7.5 L (5 vol) of water and the mixture was cooled to 30° C. Stirring was stopped and the layers were separated. The organic solution was filtered to remove particulates and distilled under reduced pressure to 3 volumes. Charged 18 L (12 vol) of water and adjust the reaction to 35° C. The reaction was seeded and stirred for 3 hours at 33-37° C. It was then cooled to 20° C. and stirred for a further 2 hours. Solids were filtered and washed twice with 4.5 L (3 vol) of water and then dried at 40° C. to give the desired title compound as white solid (2200 g, 90% yield). ¹H NMR (DMSO-d₆, 500 MHz, 25 C): 7.09 (d, J=8.6 Hz, 2H), 6.76 (d, J=8.7 Hz, 2H), 5.20 (d, J=5.1 Hz, 1H), 5.13 (d, J=7.7 Hz, 1H), 5.0 (d, J=4.7 Hz, 1H), 4.91 (d, J=5.2 Hz, 1H), 4.50 (h, J=6.0 Hz, 1H), 4.42 (t, J=5.6 Hz, 1H), 4.34 (h, J=6.9 Hz, 1H), 3.63 (ddd, J₁=1.9 Hz, J₂=5.4 Hz, J₃=11.8 Hz, 1H), 3.52 (s, 2H), 3.44-3.51 (m, 1H), 3.14-3.26 (m, 3H), 3.08-3.14 (m, 1H), 2.07 (s, 3H), 1.27 (dd, J₁=4.7 Hz, J₂=6.6 Hz, 6H), 1.22 (d, J=6.2 Hz, 6H).

What is claimed is:

1. A process for preparing a compound of formula (II), 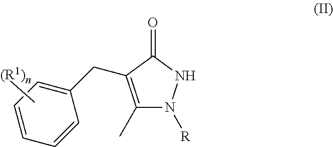
comprising the steps of:
(i) O-sulfonating a compound of formula (Ia) 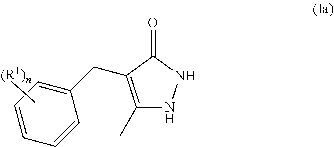
to produce a compound of formula (Ib); 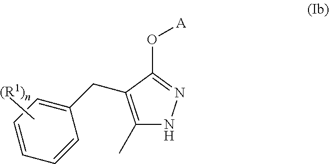
(ii) alkylating the compound of formula (Ib) to produce a compound of formula (Ic); and 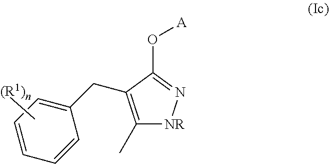
(iii) desulfonating the alkylated compound of formula (Ic) to produce the compound of formula (II);
wherein:
R is C₁-C₆ alkyl;
n is 0-3,
R¹ is C₁-C₆ alkyl, C₁-C₆ haloalkyl, C₂-C₆ alkenyl, C₂-C₆ alkynyl, C₁-C₆ acyl, C₁-C₆ alkoxy, C₁-C₆ haloalkoxy, C₁-C₆ alkylthio, C₁-C₆ haloalkylthio, C₁-C₆ alkylamino, C₃-C₇ cycloalkyl, C₃-C₇ cycloalkyloxy, or halo; and
A is a sulfonyl or sulfinyl containing hydroxyl protecting group.

2. A process as claimed in claim 1, further comprising step (iv):
(iv) reacting a compound of formula (II) with a glucose derivative to provide a compound of formula (III), 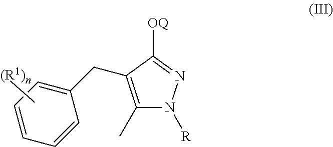
wherein Q is: [chemical structure]
